(19) United States
(12) Patent Application Publication
(10) Pub. No.: US 2025/0255962 A1
(43) Pub. Date: Aug. 14, 2025

(54) Peptide-Protected Gold Nanocluster and Uses Thereof

(71) Applicant: Queen's University at Kingston, Kingston (CA)

(72) Inventors: Parimah Aminfar, Kingston (CA); Kevin Stamplecoskie, Kingston (CA)

(21) Appl. No.: 19/051,304

(22) Filed: Feb. 12, 2025

Related U.S. Application Data
(60) Provisional application No. 63/552,337, filed on Feb. 12, 2024.

(30) Foreign Application Priority Data
Nov. 29, 2024 (CA) 3258046

(51) Int. Cl. A61K 41/00; A61K 9/08; A61K 47/62; A61K 47/69; A61P 35/00

(52) U.S. Cl. CPC A61K 41/0057; A61K 9/08; A61K 47/62; A61K 47/6923; A61P 35/00

(57) ABSTRACT

A gold nanocluster of the formula Au₂₂(Lys-Cys-Lys)₁₆, wherein Lys-Cys-Lys is lysine-cysteine-lysine, and methods for synthesis, are provided. The gold nanocluster effectively produces Type I ROS and functions as photosensitizer, and has utility in applications such as, but not limited to, biomedical applications and photocatalysis. The gold nanocluster may be useful in photodynamic therapy (PDT) and as a radiosensitizer in cells and tissues for treating diseases such as certain cancers.

### RELATED APPLICATION

This application claims the benefit of the filing date of Application No. 63/552,337, filed on Feb. 12, 2024, the contents of which are incorporated herein by reference in their entirety.

### FIELD

The invention relates to the field of nanoclusters. More specifically, the invention relates to atomically precise peptide-protected gold nanoclusters, methods for their preparation, and uses in biomedical applications and photocatalysis.

### BACKGROUND

Atomically precise gold and silver nanoclusters (NCs) have been extensively developed in recent years due to their potential for use in many areas, from catalysis to biomedical applications [1-8]. A primary motivation for using pure atomically precise samples is the structural dependence of their properties. This is particularly important in biological applications, where purity and optical/electronic characterization become crucial in their utility. A deep insight into the ground state and excited state behavior of nanoclusters, in addition to tracking them in the body and understanding how they interact with biological environments, are required.

Synthesis of nanoclusters of defined size is a significant challenge, and crude solutions of metal clusters are often complex mixtures of different sizes of nanoclusters (different numbers of metal atoms and ligands). Despite the fact that there has been progress in separation of clusters with different sizes from crude solutions, the techniques are often time-consuming and need to be well-optimized for new clusters. Furthermore, most of the purification techniques have focused on specific metal cores and ligands such as Au₂₅ and glutathione (GSH).¹⁵ Size focussing to obtain pure samples can take a week or more when isolating pure clusters. The structure/properties of individual clusters can vary greatly, and major electronic changes can occur with the exchange of even a single metal atom or ligand. For more than a decade, studies of clusters have included mixtures of similar sizes of cluster and numbers of coordinating ligands. Consequently, preparations of uniform nanoclusters and studies of their properties and utility in various applications are lacking.

### SUMMARY

One aspect of the invention relates to a nanocluster of the formula Au₂₂(Lys-Cys-Lys)₁₆; wherein Lys-Cys-Lys is lysine-cysteine-lysine.

Another aspect of the invention relates to a solution comprising: atomically precise nanoclusters of the formula Au₂₂(Lys-Cys-Lys)₁₆; and a suitable carrier; wherein Lys-Cys-Lys is lysine-cysteine-lysine. In one embodiment, substantially all of the nanoclusters in the solution are Au₂₂(Lys-Cys-Lys)₁₆.

According to embodiments, an isotope pattern corresponding to the nanoclusters exhibits an electrospray ionization mass spectrometry peak of about 1547 m/z to about 1549 m/z.

In some embodiments an isotope pattern corresponding to the nanoclusters exhibits an electrospray ionization mass spectrometry peak of about 1547.6 m/z.

In one embodiment an isotope pattern corresponding to the nanoclusters exhibits an electrospray ionization mass spectrometry peak of about 1547.57 m/z.

In some embodiments the nanoclusters exhibit an absorbance at about 500 nm and an emission at about 790 nm.

Another aspect of the invention relates to a method for preparing nanoclusters of the formula Au₂₂(Lys-Cys-Lys)₁₆, wherein Lys-Cys-Lys is lysine-cysteine-lysine, the method comprising: reacting aqueous Lys-Cys-Lys with aqueous HAuCl₄ in the presence of Omnirad 2959 as a photo-initiator at pH 11; removing oxygen and irradiating with UVA to obtain a mixture of Auₓ(Lys-Cys-Lys)_{y} nanoclusters; and size-focusing by irradiating the Auₓ(Lys-Cys-Lys)_{y} mixture with light at 620 nm to obtain atomically precise Au₂₂(Lys-Cys-Lys)₁₆ nanoclusters.

Another aspect of the invention relates to a method for providing photodynamic therapy to a subject, comprising: providing Au₂₂(Lys-Cys-Lys)₁₆ nanoclusters to the subject together with a suitable carrier, wherein cells of the subject uptake the nanoclusters; subjecting the cells to irradiation with light; wherein photosensitization and/or cell death is induced in at least a portion of the cells that uptake the Au₂₂(Lys-Cys-Lys)₁₆ nanoclusters.

In one embodiment at least a portion of the cells wherein cell death is induced are cancer cells. In one embodiment the cells are subjected to irradiation with visible light. In one embodiment, subjecting the cells to irradiation with light causes Type I ROS generation by the Au₂₂(Lys-Cys-Lys)₁₆ nanoclusters in the cells.

Another aspect of the invention relates to a method for treating cancer, comprising: providing Au₂₂(Lys-Cys-Lys)₁₆ nanoclusters to cancer cells together with a suitable carrier, wherein cancer cells uptake the nanoclusters; subjecting the cancer cells to irradiation with light; wherein cell death is induced in at least a portion of the cancer cells that uptake the Au₂₂(Lys-Cys-Lys)₁₆ nanoclusters.

Another aspect of the invention relates to a method for generating a reactive oxygen species (ROS), comprising subjecting Au₂₂(Lys-Cys-Lys)₁₆ nanoclusters to irradiation with light.

Another aspect of the invention relates to a method for photocatalyzing a chemical reaction of two or more reactants, comprising: providing a mixture of the two or more reactants and Au₂₂(Lys-Cys-Lys)₁₆; and irradiating the mixture with light; wherein the Au₂₂(Lys-Cys-Lys)₁₆ generates a reactive oxygen species that catalyzes the chemical reaction.

Another aspect of the invention relates to a photocatalyst comprising Au₂₂(Lys-Cys-Lys)₁₆.

Another aspect of the invention relates to a radiosensitizer comprising Au₂₂(Lys-Cys-Lys)₁₆.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIG. 1 is a scheme for synthesis of Au₂₂(Lys-Cys-Lys)₁₆ clusters using Omnirad 2959 as a photochemical initiator to generate reducing alpha-hydroxy radicals, followed by accelerated size-focusing of the crude mixture, according to one embodiment.

FIG. 2A is a plot of absorbance spectrum of an Auₓ(Lys-Cys-Lys)_{y} mixture of nanoclusters isolated after 12 minutes of irradiation with UVA light, along with its emission spectrum (λexc=600 nm) and excitation spectrum (λems=760 nm); inset: appearance of the product.

FIG. 2B is a plot of fluorescence EEM of the Auₓ(Lys-Cys-Lys)_{y} mixture of FIG. 2A.

FIG. 2C shows plots of fluorescence EEM of components obtained from PARAFAC analysis of the mixture of FIG. 2A.

FIG. 3A is a plot of absorbance spectra of Auₓ(Lys-Cys-Lys)_{y} mixture over 24 h LED light irradiation for size-focusing.

FIG. 3B is a fluorescence EEM plot of size-focused Au₂₂(Lys-Cys-Lys)₁₆ nanoclusters after 24 h LED irradiation.

FIGS. 3C and 3D are score plots of a three-component model of nanoclusters obtained from PARAFAC analysis during 12 min of UVA irradiation and 12 min of UVA irradiation followed by 24 h size-focusing, respectively.

FIG. 4A is a plot of absorbance spectrum and emission spectrum (λexc=500 nm) and excitation spectrum (λems=790 nm) of Au₂₂(Lys-Cys-Lys)₁₆ nanoclusters; inset: appearance of the product.3

FIG. 4B is a fluorescence EEM plot of Au₂₂(Lys-Cys-Lys)₁₆ nanoclusters.

FIG. 4C is a plot of ESI-MS data of Au₂₂(Lys-Cys-Lys)₁₆ nanoclusters obtained using a spray voltage of 4 kV in a positive ion mode, together with simulation data for Au₂₂(C₁₂H₂₂NS)₁₆H₈ (z=+5).

FIG. 4D is a structure for an Au₂₂(Lys-Cys-Lys)₁₆ nanocluster, according to one embodiment.

FIG. 5A is a Jablonski diagram of electronic excitation and excited state dynamics involved in the relaxation of Au₂₂(Lys-Cys-Lys)₁₆ clusters and ROS generation pathways, according to one embodiment.

FIG. 5B is a plot of fluorescence intensity of dichlorofluorescein (DCF) probe at 525 nm with Xe lamp irradiation in the presence of Au₂₂(Lys-Cys-Lys)₁₆ nanoclusters and of PBS blank at 488 nm excitation wavelength.

FIGS. 6A-6F are plots of (A) absorbance and (B) emission spectra of Au₂₂(Lys-Cys-Lys)₁₆ nanoclusters at various pH; (C) absorbance and (D) emission spectra monitored for 24 h in 50% FBS in PBS; (E, F) corresponding spectra in TME conditions.

FIGS. 7A-7F are plots of (A, B) cell viability after 24 h incubation with different nanocluster concentrations; (C, D) cellular uptake measured by ICP-MS after 24 h incubation with different concentrations and (E, F) at different time points for KB cells (A, C, E) and 4T1 cells (B, D, F).

FIG. 7G is a series of confocal microscopy images demonstrating cellular internalization after incubation with 10 μM nanoclusters for 3, 6 and 24 h. ****P<0.001, ***P<0.005, **P<0.01, *P<0.05.

FIGS. 8A-8D are plots of cell viability of (A) KB and (B) 4T1 cells measured by colony formation as a function of radiation dose with and (controls) without nanoclusters (20 μM concentration, 24 h incubation time); (C, D) corresponding clonogenic cell survival curves: ****P<0.001, ***P<0.005, **P<0.01, *P <0.05; the images at the top are examples of colony formation.

### DETAILED DESCRIPTION OF EMBODIMENTS

As used herein, the terms “cluster” and “nanocluster” are used interchangeably and refer to atomically precise structures in a size range smaller than nanoparticles (e.g., typically up to about 2.0 nm). Unlike nanoparticles, which are a conglomeration of materials with an average size distribution, nanoclusters are single molecules with specific molecular weights, chemical formulas, and optical properties characterized by individual electronic transitions rather than electron oscillations (plasmonics). Unlike metal nanoparticles, metal nanoclusters have well-defined molecular orbitals with discrete energy transitions and electronic structure and having size-dependent properties that can be characterized by molecular techniques such as single crystal X-ray diffraction, nuclear magnetic resonance spectroscopy, and mass spectrometry. Metal nanoclusters typically comprise a few to several hundred metal atoms and may be stabilized with ligands based on, e.g., thiols, phosphines, and halides.

As used herein, the term “atomically precise” nanoclusters refers to nanoclusters that have a selected number of metal atoms and a selected number of ligands, regardless of the exact atomic position of all the atoms and ligands. In a quantity of atomically precise nanoclusters, the predominant nanocluster has the selected number of metal atoms and the selected number of ligands, resulting in the quantity of nanoclusters having one or more selected properties. A quantity of atomically precise nanoclusters may include some nanoclusters having other numbers of metal atoms and/or other numbers of ligands, but such other nanoclusters are not present to the extent that the one or more selected properties are significantly degraded.

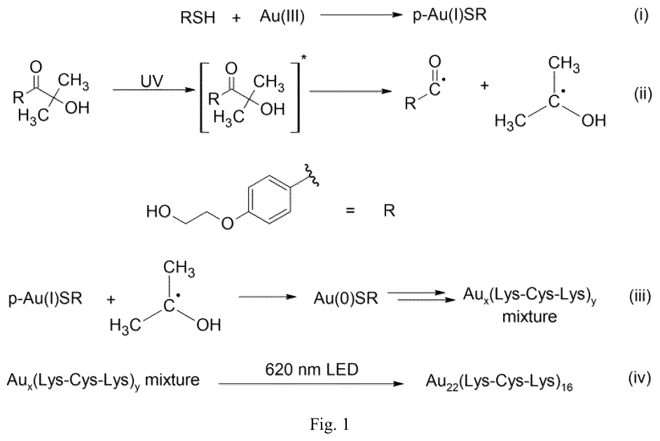

Described herein are atomically precise emissive peptide-protected gold nanoclusters and their synthesis and characterization. For the synthesis, Omnirad 2959 (IGM Resins USA Inc., Charlotte, NC, U.S.A.) was used as a photochemical reducing agent in the presence of lysine-cysteine-lysine (Lys-Cys-Lys) tripeptide as a ligand. This photochemical route avoided the problem of polydispersity of clusters, which is often unavoidable when using typical reducing agents such as NaBH₄. A rapid size-focusing step was developed and used in the synthesis to isolate the most stable structure, Au₂₂(Lys-Cys-Lys)₁₆. During synthesis, formation of Au₂₂(Lys-Cys-Lys)₁₆ nanoclusters was monitored using absorbance spectroscopy and fluorescence EEM spectroscopy. PARAFAC analysis was used to determine the number of intermediates and track their formation during the synthesis. The rapid size-focusing step was performed by irradiating the crude mixture with light at 620 nm for 24 h, providing atomically precise and stable Au₂₂(Lys-Cys-Lys)₁₆ clusters (see scheme in FIG. 1).

The Lys-Cys-Lys motif has been previously used to synthesize highly luminescent Ag nanoclusters [9]. This cationic tripeptide is highly water soluble and facilitates cellular uptake due to the positively charged Lys unit under neutral pH, favorable for many biomedical applications. Using Lys-Cys-Lys ligand and a synthesis using NaBH₄ as a reducing agent, Wang et al. [19] reported a mixture of different sizes (different numbers of metal atoms and ligands) of red-fluorescent Au nanoclusters that can stain nucleoli for bio-imaging. In contrast, provided herein are atomically precise nanoclusters (i.e., having predominantly the same numbers of Au atoms and ligands) using this ligand, which are critical to further studies and advancing to real applications in medicine. The inventors are not aware of any other reports of atomically precise Au clusters using this ligand.

Fluorescence EEM spectroscopy and PARAFAC analysis were used to support the formation of atomically precise emissive clusters during the synthesis and size focusing for optical purity of the final product. The assignment of Au₂₂(Lys-Cys-Lys)₁₆ was supported by high resolution Electrospray Ionization Mass Spectrometry (ESI-MS). The excited state reactivity and energy transfer capability of Au₂₂(Lys-Cys-Lys)₁₆ clusters were then studied using chemical probes specific for Type I and Type II reactive oxygen species (ROS) generation.

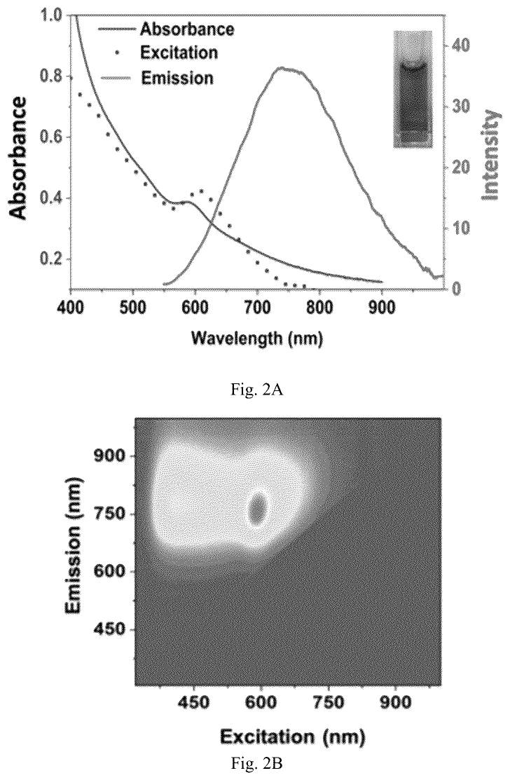

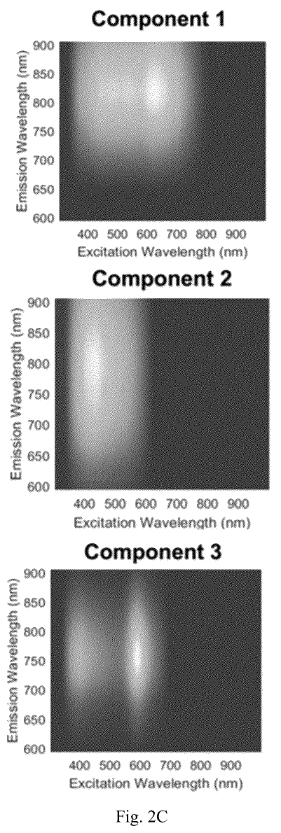

To synthesize Au₂₂(Lys-Cys-Lys)₁₆, a 9 mM aqueous solution of Lys-Cys-Lys was first reacted with aqueous HAuCl₄ (3 mM) in the presence of 9 mM Omnirad 2959 as a photo-initiator. 1 mM NaOH was added to adjust the pH of clear solution to 11. To remove oxygen, the solution was subsequently purged with nitrogen gas for 15 minutes in a quartz cuvette. The solution was then irradiated with 5 UVA lamps (250 Wm⁻²). Absorbance and fluorescence EEM spectra of the solution were monitored during irradiation. After 12 minutes of irradiation, a mixture of Auₓ(Lys-Cys-Lys)_{y} NCs was formed with an absorbance feature at 600 nm and emission at 760 nm (FIG. 2A). Fluorescence EEM of the Auₓ(Lys-Cys-Lys)_{y} mixture is shown in FIG. 2B. Fluorescence EEM spectroscopy (FIG. 2B) followed by PARAFAC analysis (FIG. 2C) revealed the formation of three emissive clusters during UVA irradiation. As expected, excitation scans of the solution did not match its absorbance spectrum, indicating a mixture of fluorescent clusters.

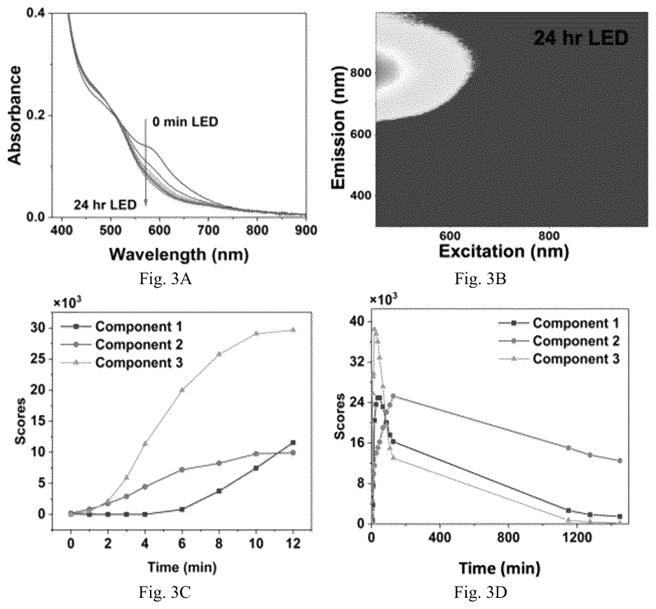

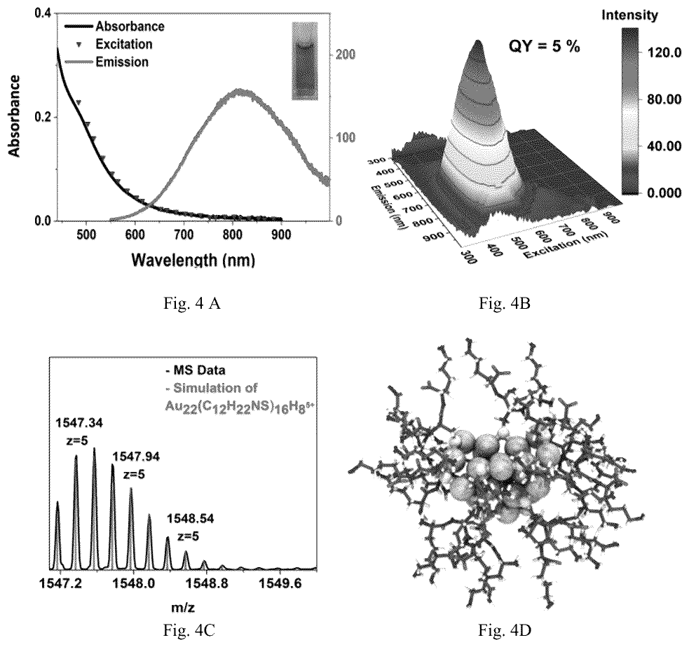

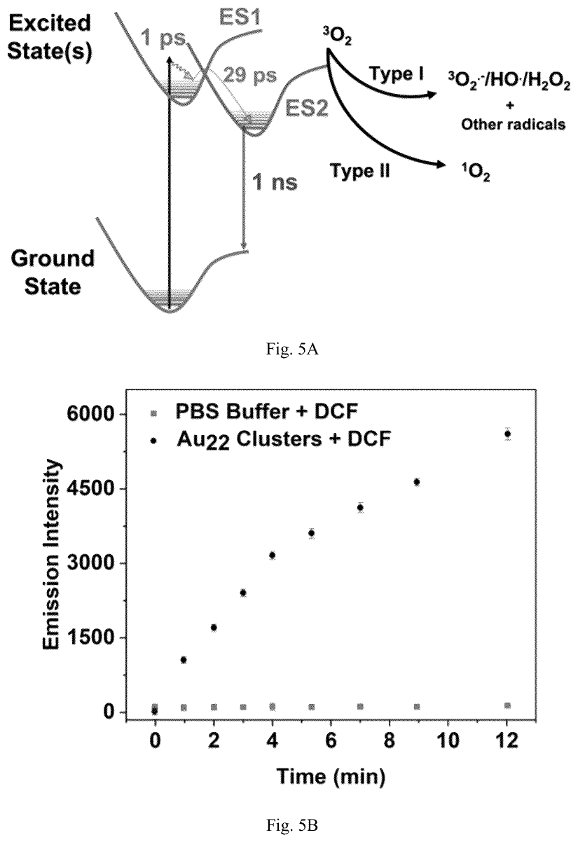

The light activated synthesized mixture was kept at 4° C. and the absorbance and fluorescence EEM spectra of the sample were monitored over the course of two weeks. Over this period the absorbance and fluorescence EEM spectrum of the mixture changed, suggesting the crude mixture was going through a slow size-focusing toward the formation of a more stable structure. To explore this, accelerated size-focusing using intense LED light was employed to accelerate the process of generating atomically precise clusters. To perform accelerated size-focusing, the solution mixture was irradiated using a 620 nm LED illuminator, 1.5 mW (Luzchem Research Inc., Ottawa, ON, Canada) and the absorbance spectrum was monitored during irradiation (FIG. 3A). After 24 h the most stable cluster, Au₂₂(Lys-Cys-Lys)₁₆, was identified with an absorbance feature at 500 nm and strong emission at 790 nm (FIG. 3B). The work-up of Au₂₂(Lys-Cys-Lys)₁₆ clusters was performed by concentrating and purifying samples by centrifugation using centrifugal filters with a 3 kDa cut-off. Au₂₂(Lys-Cys-Lys)₁₆ clusters were then stored in a refrigerator where they were stable for more than a month at 4° C.

In the first step of synthesis of Au₂₂(Lys-Cys-Lys)₁₆ clusters, thiol containing tripeptide solution reduced Au(III) to Au(I) to form thiol coordinated intermediate Au(I)-SR complexes. The second step of the synthesis involved formation of alpha hydroxy radicals upon UVA irradiation and reduction of Au(I)-SR species to form the metal cores (FIG. 1, equation (ii)). In the final step, size-focusing was performed using LED irradiation, providing the most stable cluster for 24 h (FIG. 1, equation (iv)). During the synthesis, the fluorescence EEM spectrum of the reaction solution was monitored every 30 seconds. Afterwards, PARAFAC analysis was performed on EEM spectra to qualitatively indicate the number of emissive components in EEM data. Before performing PARAFAC analysis, the scattering contribution was removed from each EEM spectra of the data set using EZspec software (Horiba Ltd., Kyoto, Japan), and each EEM was subsequently normalized and smoothed using MATLAB (The Math Works, Inc., Natick, MA, U.S.A.). Prior to mathematical decomposition, the number of components that best described the dataset were approximated. PARAFAC analysis was then performed on the EEM scans using drEEM Toolbox for MATLAB. The best fit to the dataset was obtained using three significant components when constraining the components to be non-negative in all dimensions. The fluorescence EEM plots generated by PARAFAC were found to describe 99.1% of the data when fit to the three components model. The components were then used to calculate the scores for the components, as the product of the component matrix and the mode matrix, which contains the weighting factors for each mode. Score values may be used as a proxy for relative concentration multiplied with the quantum yield. The fluorescence EEM spectra of the three components overlap considerably, and were suitably subjected to multivariate analysis.

Score values obtained from PARAFAC model are proportional to the concentration of each component. From the score plot, it can be observed that after performing accelerated size-focusing (620 nm LED irradiation overnight), component 2 was the most abundant emitter in the solution (FIGS. 3C and 3D). Additionally, the nanocluster purity was highest using a shorter UVA irradiation time of the crude mixture (12 min) rather than a longer irradiation time (30 min UVA).

Concentration of precursors and pH of the solution are two factors that may influence which material is isolated in light activated synthesis of metal clusters. The concentration of reducing agent (Omnirad 2959) played an important role in formation of the Auₓ(Lys-Cys-Lys)_{y} mixture. The best concentration of reducing agent was found to be three times higher than initial concentration of HauCl₄ salts, for reducing the Au(III) ions to Au(0). Notedly, with lower concentrations of Omnirad 2959 no species emitting above 600 nm formed even after 1 h of UVA irradiation. Higher concentrations of reducing agent caused inner-filter effects which slow the reduction step (necessary for formation of clusters rather than nanoparticles) by lowering the steady state concentration of reducing radicals, thus forming NIR emitting clusters. As the reduction of Au(I)-SR intermediates is pH dependent (FIG. 1, equation (i)), the pH of Au(I)-SR containing solution was adjusted to five different values (8-12) prior to UVA irradiation. The highest yield for the product was obtained at pH 11 as determined from absorbance after 12 min of UVA irradiation.

The effect of non-linear processes on Au₂₂(Lys-Cys-Lys)₁₆ production was investigated. For example, if only the photon flux (concentration) was important, there would be no difference in yield if the photons were irradiated continuously or in short, intense pulses with UVA light. The Au(I)-SR reaction solutions were irradiated with 340 nm pulsed laser light (˜1 nJ/pulse, with 250 fs FWHM). After 11 h of pulsed laser irradiation the total deposited energy was identical to that of continuous irradiation for 1 min. It was found that absorbance of the product was similar to the absorbance profile of nanoclusters formed after 1 min of UVA irradiation, suggesting non-linear effects do not play an important role in the synthesis.

To assess the thermal effects of irradiation a thermal reduction method was studied with Lys-Cys-Lys ligand. The Au(I)-SR intermediates were heated to 55° C. and after 24 h blue emitting clusters were formed. These were similar to the products obtained from light activated synthesis when low concentration of the photochemical reducing agent was used, as opposed to Au₂₂(Lys-Cys-Lys)₁₆.

The emission quantum yield of Au₂₂(Lys-Cys-Lys)₁₆ was found to be 5%, measured with a Hamamatsu absolute quantum yield spectrometer. TEM imaging of Au₂₂(Lys-Cys-Lys)₁₆ solution confirmed the presence of particles that were significantly smaller than 2 nm (˜1.66 nm). The absorbance spectrum of Au₂₂(Lys-Cys-Lys)₁₆ and its emission spectrum (λexc=500 nm) and excitation spectrum (λems=790 nm) are shown in FIG. 4A, and the inset in the figure shows the appearance of the product. A fluorescence EEM plot of Au₂₂(Lys-Cys-Lys)₁₆ is shown in FIG. 4B.

The assignment of Au₂₂(Lys-Cys-Lys)₁₆ using mass spectrum of atomically precise clusters was obtained in positive ion mode in ESI-MS (FIG. 4C). The range was about 1547-1549 m/z with a peak at about 1547.6 m/z (1547.57 m/z). The spectrum is well matched with that of Au₂₂(C₁₂H₂₂NS)₁₆ H₈ (z=+5) which corresponds to Au₂₂(Lys-Cys-Lys)₁₆ in solution (e.g., in a carrier such as water). As a result of ligand fragmentation, only a portion of the ligand remains attached to the metal core. Similar fragmentation patterns have been observed for captopril and glutathione protected Au₁₈ clusters [10].

A three-dimensional non-spherical model was developed for the structure of Au₂₂(C₁₂H₂₂NS)₁₆ clusters (FIG. 4D) using density functional theory (DFT as implemented in software GPAW and Perdew-Burke-Ernzerhof (PBE) exchange correlation functional [12]. The published crystal structure for the metal core of Au₂₂(SAdm)₁₆ was used as a starting point and the SAdm thiolate ligands were replaced with Lys-Cys-Lys ligands. Bond angles and directions at the metal-ligand interface were modified as necessary to avoid overlaps between neighbouring ligands. The initial structures were first optimized by the ligand layer to avoid artifacts due to the non-optimal initial arrangement of the organic part. Next the full structure was optimized by letting all atoms be free. The electronic structure of the model cluster was solved with three charge states 2+, neutral, and 2− which gave HOMO-LUMO gaps of 0.02 eV, 1.27 eV, and 0.83 eV respectively. The neutral charge structure was used in further modelling because it exhibited the best electronic stability. The calculated absorption spectrum of the neutral system was generally in agreement with the experimentally measured spectrum. Both show a monotonous slow increase of absorption intensity below 900 nm until the change to higher probability transitions with an onset around 600 nm. The calculated spectrum shows four identifiable features at 675 nm (low extinction), 505 nm, 450 nm and 390 nm. Hence, based on UV-Vis characterization the model structure seems plausible to explain observed spectra.

X-ray absorption spectroscopy (XAS) was used to further characterize the Au₂₂(Lys-Cys-Lys)₁₆ clusters by probing the local structure of the Au atoms in the cluster. Au L₃-edge EXAFS data was collected in transmission mode at the Sector 20-BM beamline of the Advanced Photon Source (Argonne National Laboratory in Argonne, Illinois) with an Au foil reference being measured simultaneously with all scans. Samples were analyzed in the aqueous phase at room temperature. Background subtraction and scan averaging were performed using Athena, part of the DEMETER package for EXAFS refinement [14]. EXAFS fitting and EXAFS simulation were performed using WINXAS 4.0.2 software [15]. The scattering paths and extended X-ray absorption fine structure spectrum of the DFT-calculated structure were simulated using FEFF8 computational software and compared to the experimental Au₂₂(Lys-Cys-Lys)₁₆ EXAFS data (SI 12a) [16]. To account for thermal disorder in the simulated EXAFS data of the DFT-calculated structure, an empirical Debye-Waller factor was obtained by fitting the experimental spectrum. This fitting was performed using an Au—S scattering path from the DFT-calculated structure to quantitatively examine the experimental data. The Au—S coordination number of 2.19, obtained from EXAFS fitting, is greater than the theoretical coordination number of 1.5 from the DFT-calculated structure. This suggests the significant presence of residual Au(I)-thiolate oligomer structures; these oligomers have a higher Au—S coordination number than the cluster, thereby artificially increasing the coordination number value obtained from the fit. The Au—S bond length of 2.295 Å from the EXAFS fitting was also in good agreement with both the DFT-calculated structure and other Au(I)-thiolate nanoclusters with similar semi-ring core structure [17,18]. Although the Au—Au scattering representing Au core structures were not of high enough intensity to reliably fit, the longer-range scattering peaks observed in the experimental EXAFS spectrum show good agreement with those of the DFT-calculated structure EXAFS spectrum.

To elucidate the excited state behavior of Au₂₂(Lys-Cys-Lys)₁₆, femtosecond transient absorbance spectroscopy (commonly also called pump-probe spectroscopy) was used. The technique provides valuable information in assessing excited state lifetimes and properties related to activity as a photosensitizer. It uses two laser pulses: the first, the pump, is used to excite the sample at a single wavelength and the other, the probe (a white light pulse), is delayed in time and probes changes in absorbance of the sample in the excited state. The difference spectrum (excited state absorbance minus ground state absorbance) is recorded in time, allowing for dynamics of the excited state and relaxation to the ground state to be monitored. Herein, an aqueous solution (pH=7) of Au₂₂(Lys-Cys-Lys)₁₆ clusters was excited with 250 fs FWHM pump centred at 340 nm and the transient absorption was recorded up to 5 ns after excitation. The results suggest the nanoclusters exhibit three relaxation components: (1) thermalization of hot electrons, (2) dynamics on the ˜1000 ps timescale that are attributed to formation of a reactive/emissive excited state, and (3) nanosecond to microsecond emission or reactivity from the lowest lying excited state. The Jablonski diagram of FIG. 5A illustrates the excited state behaviour of the clusters and possible reaction pathways with oxygen.

The efficacy in excited state reactivity and generation of reactive oxygen species of metal NCs is highly dependent on their exact composition. The excited state of metal clusters has been shown to be reactive with different substrates. In the case of photoinduced therapies such as photodynamic therapy (PDT), the substrate is usually oxygen and the products are ROS.

Two chemical probes were used to study ROS generation of Au₂₂(Lys-Cys-Lys)₁₆. First, the production of ¹O₂ was assessed using 9,10-anthracenediyl-bis (methylene) dimalonic acid (ABDA), which is water-soluble and specific for ¹O₂ detection. ABDA undergoes oxidation to form endoperoxide ABDAO₂ when ¹O₂ is generated by clusters, leading to loss in absorbance of ABDA at 350-400 nm. Methylene blue (MB) was used as a positive control, which is commonly used for the photochemical generation of ¹O₂ (with QY of 0.52). Using equimolar concentrations of ABDA in water, two solutions were prepared. The first solution contained 50 uL of ABDA stock solution (0.15 mg/mL) which was added to the clusters with a total solution volume of 2.6 mL, where the absorbance of the clusters and the probe were set to 0.2 at 520 nm (excitation wavelength). To the second solution, 50 uL of ABDA stock solution was added to a 2.6 mL MB solution, where the absorbance of the MB and the probe were matched at 520 nm. Using 520 nm LED light, the solutions were irradiated in 5-minute intervals up to 30 minutes and absorbance spectra were acquired at each interval. While the positive control showed the expected absorbance reduction as a result of ¹O₂ generation, no specific change was observed when using Au₂₂(Lys-Cys-Lys)₁₆ nanoclusters.

The second probe, 2,7-dichlorodihydrofluorescein (DCFH) was used to assess Type I ROS generation of the clusters. DCFH is sensitive to radicals such as H₂O₂, HO, and ROO. Non-fluorescent DCFH can be oxidized by ROS and form dichlorofluorescein (DCF), which is emissive at 525 nm. For this experiment, 250 mL of DCFH ethanol solution (1 mM) was added to the 1 mL sodium hydroxide solution (0.01 mM) which was subsequently diluted with 5 mL of PBS buffer in the dark to hydrolyze for 30 minutes. 200 mL of the Au₂₂(Lys-Cys-Lys)₁₆ clusters were then added to 1 mL of DCFH solution and irradiated with a Xe lamp (100 m W cm⁻²) for 1 min intervals. The fluorescence spectra were measured at 488 nm excitation wavelength. An increase in the fluorescent maxima of the DCF probe was observed over 12 min of irradiation, suggesting that the excited state of Au₂₂(Lys-Cys-Lys)₁₆ clusters is reactive and Type I ROS has been generated (FIG. 5B). The excited state reactivity of Au₂₂(Lys-Cys-Lys)₁₆ clusters makes them a suitable candidate for use in photocatalysis. In addition, the ROS generation capability of Au₂₂(Lys-Cys-Lys)₁₆ clusters indicates their potential to function as photosensitizers and thus they are expected to have utility in applications such as, but not limited to, biomedical applications and photocatalysis. For example, the nanoclusters may be useful in photodynamic therapy (PDT), which may include providing Au₂₂(Lys-Cys-Lys)₁₆ nanoclusters to a subject together with a suitable carrier, wherein cells of the subject uptake the nanoclusters. The cells are then irradiated with light which causes photosensitization of the cells and/or cell death in at least a portion of the cells that uptake the Au₂₂(Lys-Cys-Lys)₁₆ nanoclusters. Some embodiments may include treating diseases such as certain cancers, and may include additives, therapeutic compounds, etc., with the Au₂₂(Lys-Cys-Lys)₁₆ nanoclusters. For example, additives may be used to enhance uptake of the nanoclusters by target cells for PDT, such as cancer cells. Furthermore, since the Au₂₂(Lys-Cys-Lys)₁₆ nanoclusters are highly photostable, they are ideal for use in biological imaging and sensing. For example, their bright and stable fluorescence can be used for in vivo imaging of cells and tissues, as well as detecting various analytes, such as metal ions and biomolecules.

The same photocatalytic activity (excited state reactivity) to form ROS that makes the nanoclusters useful for radiation therapies is a good illustration of the photocatalytic activity of the excited state. This photocatalytic activity can be used to convert reagents to high value products making Au₂₂(Lys-Cys-Lys)₁₆ nanoclusters excellent candidates for catalyzing innumerable reactions and transformations. The extensive field of photo-redox catalysis, for example, may see utility in Au₂₂(Lys-Cys-Lys)₁₆ nanoclusters as a new photocatalyst.

The contents of all cited documents are incorporated herein by reference in their entirety.

Embodiments are further described by way of the following non-limiting examples.

### EXAMPLE 1

### Stability of Nanoclusters Under Physiological Conditions

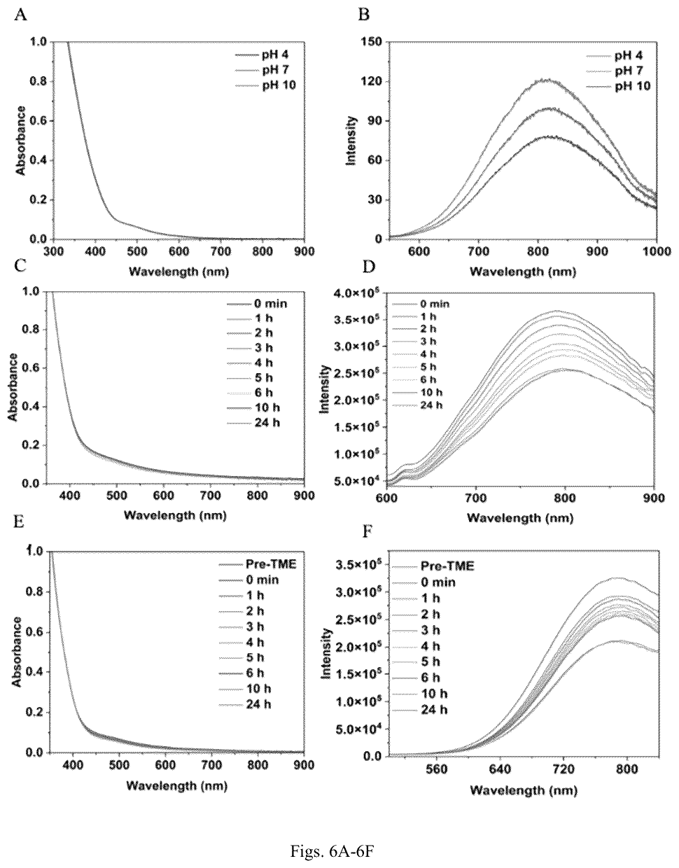

UV-Vis and emission spectroscopy were used to monitor the stability of the nanoclusters under various conditions that simulate physiological microenvironments. FIGS. 6A-6F show the optical stability under varying pH and serum conditions, and in a tumor microenvironment (TME) model to evaluate potential degradation or alterations. As shown in FIG. 6A, the absorbance profile of the nanocluster remained stable across a broad pH range of 4-10. However, as shown in FIG. 6B, under both acidic (pH 4, lower curve) and basic (pH 10, middle curve) conditions, a slight decrease in fluorescence intensity was observed, while the overall absorption and emission spectra remained largely unchanged. Thus, the nanoclusters retain structural stability across a wide pH range, with the minor change in fluorescence intensity likely due to varying protonation states of the Lys-Cys-Lys peptide ligand at different pH.

The nanocluster stability in a serum-mimicking environment (50% fetal bovine serum, FBS, in phosphate buffered saline, PBS) was evaluated through real-time monitoring of its absorption and fluorescence spectra. As shown in FIGS. 6C and 6D no significant changes were observed except for a gradual reduction in fluorescence intensity over time, which remained >70% after 24 h incubation. This stability is suitable for in vivo applications.

The tumor microenvironment (TME) exhibits unique chemical conditions, including mild acidity, elevated hydrogen peroxide levels and hypoxia. To simulate these conditions in solution, the NCs were incubated in a buffer mimicking the TME (pH 6.5 and 10 μM H₂O₂). The absorption and emission spectra remained substantially unchanged, with 70% of the initial fluorescence intensity retained after 24 h incubation (FIGS. 6E and 6F).

### EXAMPLE 2

### Dark Toxicity and Intracellular Uptake in Cancer Cells In Vitro

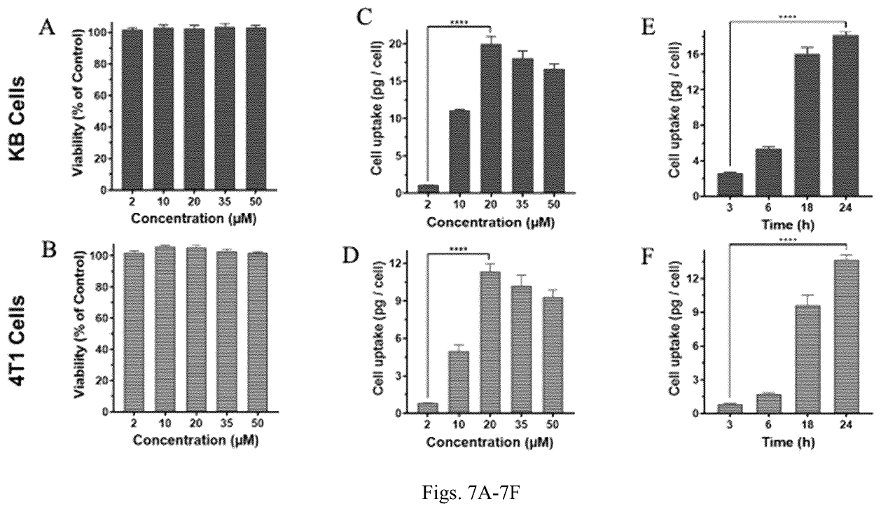

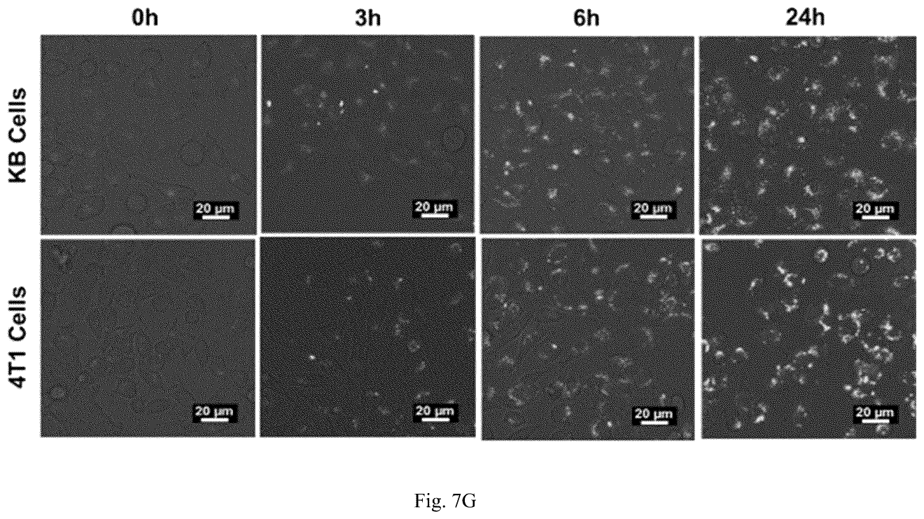

The dark cytotoxicity of the NCs was evaluated using the AlamarBlue assay in two cancer cell lines, human-derived KB and murine breast cancer 4T1, over 24 h incubation at concentrations ranging from 2 to 50 μM. No significant cytotoxicity was observed (FIGS. 7A and 7B).

The intracellular uptake measured by inductively-coupled mass spectrometry (ICP-MS) in KB and 4T1 cells after incubation in 2-50 μM NCs for 24 h demonstrated well-correlated concentration-dependent increases in intracellular uptake at range of 2-20 μM for both cell lines with over 10 times enhanced cell uptake at 20 μM versus at 2 μM (P<0.0001) (FIGS. 7C and 7D). Thereafter, there was a plateau in uptake. These findings suggest active uptake mechanism(s) with saturation at higher concentrations. Additionally, time-dependent increases in uptake were observed when incubating cells with a fixed concentration of 20 μM for 3, 6, 18, and 24 h, with significantly higher accumulation at 24 h compared to 3 h incubation (P<0.0001) (FIGS. 7E and 7F).

The observed intracellular concentration of gold was >10 pg per cell, which is significantly greater than that reported for AuNCs with either cyclic peptide ligand, cyclic arginine-glycine-aspartic acid-D-tyrosine-cysteine (c(RGDyC)) (<1 pg per cell) [20] or Falate (FA) ligand (<0.04 pg per cell) [21]. This high uptake is likely due to the positively-charged Lys-Cys-Lys tripeptide framework, which interacts favorably with the negatively-charged cell membrane. The intrinsic fluorescence of Au₂₂(Lys-Cys-Lys)₁₆ NCs also enabled tracking of intracellular uptake via confocal microscopy, confirming efficient and time-dependent internalization (FIG. 7G).

### EXAMPLE 3

### Efficacy of Au₂₂(Lys-Cys-Lys)₁₆ as a Radiosensitizer

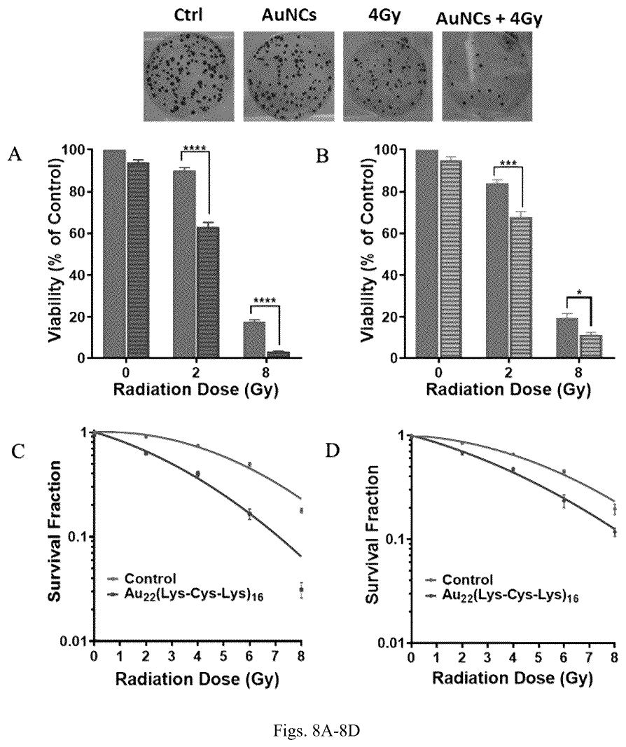

Given that the core of the Au₂₂(Lys-Cys-Lys)₁₆ nanoclusters is composed of multiple gold atoms capable of strong interactions with ionizing radiation, combined with its favorable biocompatibility and efficient intracellular uptake, this nanocluster is expected to serve as an effective radiosensitizer. Colony-forming assay after 24 h incubation and exposure to 2, 4, 6, or 8 Gy of 225 kVp X-irradiation were performed, with no-treatment, NC-only, and radiation-only as controls. Au₂₂(Lys-Cys-Lys)₁₆ showed no cytotoxicity but demonstrated enhanced cell death at all radiation doses in both KT and 4T1 cell lines (FIGS. 8A and 8B, respectively). At 2 Gy, the nanocluster reduced KB and 4T1 cell survival to 65% and 64%, respectively, compared to 87% and 82% in the radiation-only groups. At 8 Gy, the nanocluster further reduced survival to ˜5% for KB cells and 10% for 4T1 cells, respectively, compared to 17% and 19% for radiation alone.

The corresponding survival curves are presented in FIGS. 8C and 8D. The dose enhancement factor (DEF) was calculated as the ratio of the dose required for 50% survival without and with Au₂₂(Lys-Cys-Lys)₁₆. The values were 2±0.25 and 1.6±0.1 for the KB and 4T1 cells (FIGS. 8C and 8D, respectively), which may be due in part to the higher NC accumulation KB cells (20 pg/cell) compared to 4T1 cells (11.4 pg/cell). The radiosensitization effect of gold nanoparticles and nanoclusters is generally influenced by multiple factors, including size, shape, surface chemistry, and concentration, which regulating intracellular uptake, and radiation type. The DEFs reported here highlight the importance of the ultra-small size (2.1 nm) and biocompatible tripeptide framework of the nanoclusters that contributes to effective intracellular uptake.

### EQUIVALENTS

It will be appreciated that modifications may be made to the embodiments described herein without departing from the scope of the invention. Accordingly, the invention should not be limited by the specific embodiments set forth but should be given the broadest interpretation consistent with the teachings of the description as a whole.

### REFERENCES

1. G. S. a. N. T. B. Corain, Elsevier, 2011.
2. A. Corma, Cluster catalysis: A subtle form of recognition, Nature Nanotechnology, 2014, 9, 412-413.
3. Y. Zhu, H. Qian, A. Das, R. Jin, Comparison of the catalytic properties of 25-atom gold nanospheres and nanorods, Chinese Journal of Catalysis, 2011, 32, 1149-1155.
4. Z. Wu, D.-e. Jiang, A. K. P. Mann, D. R. Mullins, Z.-A. Qiao, L. F. Allard, C. Zeng, R. Jin, S. H. Overbury, Thiolate Ligands as a Double-Edged Sword for CO Oxidation on CeO2 Supported Au25(SCH₂CH₂Ph)₁₈ Nanoclusters, Journal of the American Chemical Society, 2014, 136, 6111-6122.
5. Y.-C. Shiang, C.-C. Huang, W.-Y. Chen, P.-C. Chen, H.-T. Chang, Journal of Materials Chemistry, 2012, 22, 12972-12982.
6. Y. Zhang, C. Zhang, C. Xu, X. Wang, C. Liu, G. I. N. Waterhouse, Y. Wang, H. Yin, Talanta, 2019, 200, 432-442.
7. D. Su, L. Gao, F. Gao, X. Zhang, X. Gao, Chemical Science, 2020, 11, 5614-5629.
8. J. Li, J.-J. Zhu, Kai Xu, Fluorescent metal nanoclusters: From synthesis to application, TrAC Trends in Analytical Chemistry, 2014, 58, 90-98.
9. N. Goswami, K. Zheng, J. Xie, Nanoscale, 2014, 6, 13328-13347.
10. G. Yousefalizadeh, K. G. Stamplecoskie, The Journal of Physical Chemistry A, 2018, 122, 7014-7022.
11. J. Enkovaara, et al., Journal of Condensed Matter Physics, 2010, 22, 253202.
12. J. P. Perdew, K. Burke, M. Ernzerhof, Physical Review Letters, 1996, 77, 3865-3868.
13. Q. Li, S. Yang, T. Chen, S. Jin, J. Chai, H. Zhang, M. Zhu, Nanoscale, 2020, 12, 23694-23699.
14. B. Ravel, M. Newville, Journal of Synchrotron Radiation, 2005, 12, 537-541.
15. T. Ressler, Journal of Synchrotron Radiation, 1998, 5, 118-122.
16. A. L. Ankudinov, B. Ravel, J. J. Rehr, S. D. Conradson, Physical Review B, 1998, 58, 7565-7576.
17. D. M. Chevrier, M. A. MacDonald, A. Chatt, P. Zhang, Z. Wu, R. Jin, The Journal of Physical Chemistry C, 2012, 116, 26947-26947.
18. Y. Yu, Z. Luo, D. M. Chevrier, D. T. Leong, P. Zhang, D.-E. Jiang, J. Xie, Journal of the American Chemical Society, 2014, 136, 1246-1249.
19. X. Wang, Y. Wang, H. He, X. Ma, Q. Chen, S. Zhang, B. Ge, S. Wang, W. M. Nau, F. Huang, ACS Appl. Mater. Interfaces, 2017, 9, 17799-17806.
20. Liang, G., et al., RGD peptide-modified fluorescent gold nanoclusters as highly efficient tumor-targeted radiotherapy sensitizers. Biomaterials, 2017. 144: p. 95-104.
21. Kefayat, A., et al., Ultra-small but ultra-effective: Folic acid-targeted gold nanoclusters for enhancement of intracranial glioma tumors' radiation therapy efficacy. Nanomedicine: Nanotechnology, Biology and Medicine, 2019. 16: p. 173-184.

1. A nanocluster of the formula Au₂₂(Lys-Cys-Lys)₁₆;
wherein Lys-Cys-Lys is lysine-cysteine-lysine.

2. A solution comprising:
atomically precise nanoclusters of the formula Au₂₂(Lys-Cys-Lys)₁₆; and
a suitable carrier;
wherein Lys-Cys-Lys is lysine-cysteine-lysine.

3. The solution of claim 2, wherein an isotope pattern corresponding to the nanoclusters exhibits an electrospray ionization mass spectrometry peak of about 1547 m/z to about 1549 m/z.

4. The solution of claim 2, wherein an isotope pattern corresponding to the nanoclusters exhibits an electrospray ionization mass spectrometry peak of about 1547.6 m/z.

5. The solution of claim 2, wherein an isotope pattern corresponding to the nanoclusters exhibits an electrospray ionization mass spectrometry peak of about 1547.57 m/z.

6. The solution of claim 2, wherein the nanoclusters exhibit an absorbance at about 500 nm and an emission at about 790 nm.

7. A method for preparing nanoclusters of the formula Au₂₂(Lys-Cys-Lys)₁₆, wherein Lys-Cys-Lys is lysine-cysteine-lysine, the method comprising:
reacting aqueous Lys-Cys-Lys with aqueous HAuCl4 in the presence of Omnirad 2959 as a photo-initiator at pH 11;
removing oxygen and irradiating with UVA to obtain a mixture of Auₓ(Lys-Cys-Lys)_{y} nanoclusters; and
size-focusing by irradiating the Auₓ(Lys-Cys-Lys)_{y} mixture with light at 620 nm to obtain atomically precise Au₂₂(Lys-Cys-Lys)₁₆ nanoclusters.

8. A method for providing photodynamic therapy to a subject, comprising:
providing Au₂₂(Lys-Cys-Lys)₁₆ nanoclusters to the subject together with a suitable carrier, wherein cells of the subject uptake the nanoclusters;
subjecting the cells to irradiation with light;
wherein photosensitization and/or cell death is induced in at least a portion of the cells that uptake the Au₂₂(Lys-Cys-Lys)₁₆ nanoclusters.

9. The method of claim 8, wherein at least a portion of the cells wherein cell death is induced are cancer cells.

10. The method of claim 8, wherein the cells are subjected to irradiation with visible light.

11. The method of claim 8, wherein subjecting the cells to irradiation with light causes Type I ROS generation by the Au₂₂(Lys-Cys-Lys)₁₆ nanoclusters in the cells.

12. A method for treating cancer, comprising:
providing Au₂₂(Lys-Cys-Lys)₁₆ nanoclusters to cancer cells together with a suitable carrier, wherein cancer cells uptake the nanoclusters;
subjecting the cancer cells to irradiation with light;
wherein cell death is induced in at least a portion of the cancer cells that uptake the Au₂₂(Lys-Cys-Lys)₁₆ nanoclusters.

13. A method for generating a reactive oxygen species (ROS), comprising subjecting Au₂₂(Lys-Cys-Lys)₁₆ nanoclusters to irradiation with light.

14. A method for photocatalyzing a chemical reaction of two or more reactants, comprising:
providing a mixture of the two or more reactants and Au₂₂(Lys-Cys-Lys)₁₆; and
irradiating the mixture with light;
wherein the Au₂₂(Lys-Cys-Lys)₁₆ generates a reactive oxygen species that catalyzes the chemical reaction.

15. A photocatalyst comprising Au₂₂(Lys-Cys-Lys)₁₆.

16. A radiosensitizer comprising Au₂₂(Lys-Cys-Lys)₁₆.
